(19) United States
(12) Patent Application Publication
(10) Pub. No.: US 2020/0365269 A1
(43) Pub. Date: Nov. 19, 2020

(54) SYSTEM FOR ALERTING TO SKIN CONDITIONS

(71) Applicant: International Business Machines Corporation, Armonk, NY (US)

(72) Inventors: Uri Kartoun, Cambridge, MA (US); Sophie Batchelder, Lancaster, PA (US); Veronica Aldous, Davie, FL (US); Fang Lu, Billerica, MA (US); Francis Campion, Westwood, MA (US)

(21) Appl. No.: 16/411,470

(22) Filed: May 14, 2019

(51) Int. Cl. G16H 50/20; G16H 70/60; G06N 5/02

(52) U.S. Cl. CPC G16H 50/20; G06N 5/02; G16H 70/60

(57) ABSTRACT

A data analysis system is configured for alerting to the results of a skin condition assessment. The data analysis system has an extraction system for extracting one or more indicators and one or more outcomes related to a plurality of skin conditions from electronic medical records. The data analysis system also has a machine learning system for generating a predictive model for each of the plurality of skin conditions based on the extracted one or more indicators and one or more outcomes. The data analysis system further has an evaluation system for receiving medical data for an individual patient and applying each predictive model to the medical data for the individual patient, and an alerting system for providing results of each predictive model to a user interface of an end-user device, wherein the results include an assessment of the likelihood that the individual patient will develop each skin condition.

### TECHNICAL FIELD

The present application relates generally to generating results from electronic medical records and, more particularly, to a system for alerting to skin conditions and vulnerabilities based on data analysis of EMRs.

### BACKGROUND

There are various different types of skin diseases (for instance, Darier's Disease, Lichen Planus Actinicus, Polymorphic Light Eruption, etc.). Many of these diseases are associated with symptoms that could be aggravated by sun exposure. Many individuals who may have one or more of these skin conditions may be unaware of their disease, and unaware of the importance of reducing sun exposure. Therefore, it would be beneficial to be able to obtain an assessment of the likelihood of having or developing a skin condition even before symptoms are present so that positive steps towards addressing the potential condition can be taken at an early stage.

### SUMMARY

In some embodiments, a computer-implemented method for alerting to the results of a skin condition assessment in a data processing system is comprising a processing device and a memory comprising instructions which are executed by the processor is disclosed. The method includes receiving medical history information for a plurality of patients, generating a predictive model for a skin condition based on the medical history information for a plurality of patients, receiving medical data for an individual patient, applying the predictive model for the skin condition to the medical data for the individual patient, and alerting to results of the predictive model by providing the results to a user interface of an end-user device, wherein the results include an assessment of the likelihood that the individual patient will develop the skin condition.

In other embodiments, a data analysis system for alerting to the results of a skin condition assessment is disclosed. The data analysis system includes an extraction system for extracting one or more indicators and one or more outcomes related to a plurality of skin conditions from electronic medical records containing medical history information for a plurality of patients, a machine learning system for generating a predictive model for each of the plurality of skin conditions based on the extracted one or more indicators and one or more outcomes, an evaluation system for receiving medical data for an individual patient and applying each predictive model to the medical data for the individual patient, and an alerting system for providing results of each predictive model to a user interface of an end-user device, wherein the results include an assessment of the likelihood that the individual patient will develop each skin condition.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 depicts a block diagram of an exemplary healthcare data environment, consistent with disclosed embodiments;

FIG. 2 is a block diagram of an example data processing system in which aspects of the illustrative embodiments are implemented;

FIG. 3 is a block diagram of an exemplary data analysis system, consistent with disclosed embodiments;

FIG. 4 is a flowchart of an exemplary process for alerting to skin conditions and vulnerabilities, consistent with disclosed embodiments; and

FIG. 5 is an example of a user interface for providing an alert regarding skin conditions and vulnerabilities, consistent with disclosed embodiments.

### DETAILED DESCRIPTION OF EXEMPLARY EMBODIMENTS

Embodiments of the present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a head disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network (LAN), a wide area network (WAN) and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The present disclosure relates to the use of EMRs and the medical data that is stored therein. EMRs may be a compilation of all information that has been recorded and stored in one or more locations in relation to a patient. An example EMR may contain demographic information, allergies, diagnoses, vital sign information, medications prescribed and taken, laboratory tests conducted and the results, operations, providers and physicians visited, physical examination records, pathology reports, clinical narrative notes, discharge summaries, radiology reports, cardiology reports, and encounter information. The medical data may be a representation of a patient's medical history stored in an EMR.

The EMR may be organized or unorganized and contained structured and/or unstructured data. An organized EMR may contain metadata and categorized information that indicates that a software program can identify the stored information. An unorganized EMR may contain the information without software being able to identify what the information represents. Structured data may be stored in tables and may include laboratory observations, comorbidities, prescriptions, dates of birth, genders, etc. that are easily recognizable and extractable as medical data. Unstructured data may include notes such as clinical narrative notes that are narrative in form and may need further review and processing to extract information.

The present disclosure relates to a data analysis system with an alerting system for developing a predictive model associated with a skin condition, assessing an individual patient using the predictive model, and providing alerts. The data analysis system may leverage available information in EMRs, including structured and unstructured data, to extract variables that can be used to train a machine learning algorithm for assessing a potentially increased risk for skin conditions in the level of the individual patient. The data analysis system may be configured to produce a result that indicates a likelihood that the patient will develop a skin condition, so that the patient can take specific steps to address high risk conditions, such as avoiding excessive sun exposure. Eosinophilic Fasciitis, for example, is a rare disorder characterized by inflammation of the tough band of fibrous tissue beneath the skin (fascia). Affected individuals are known to have elevated levels of certain white blood cells (eosinophils), a commonly available biomarker in any EMR-based health care system.

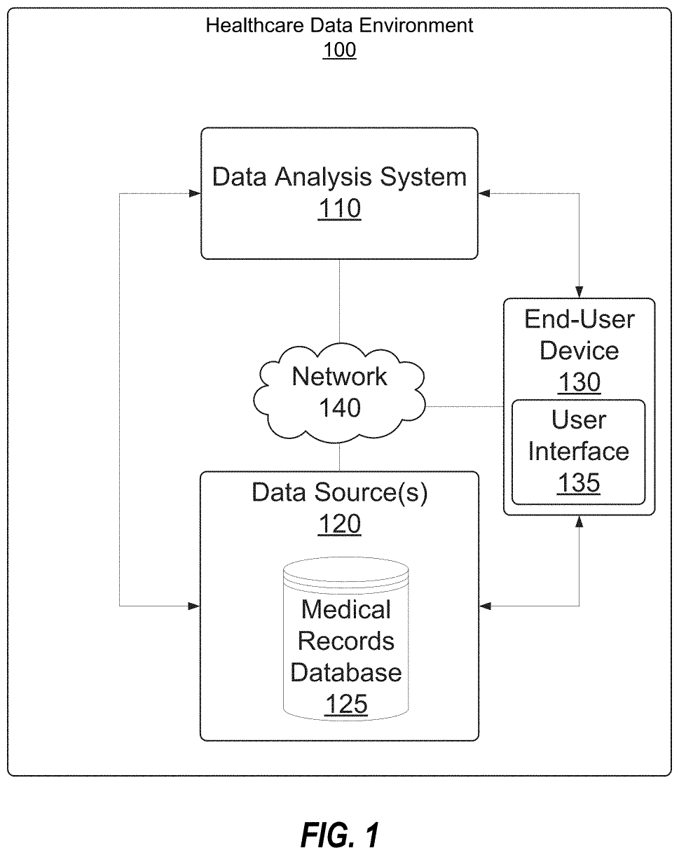

FIG. 1 is an illustration of an exemplary healthcare data environment 100. The healthcare data environment 100 may include a data analysis system 110, one or more data sources 120, and an end-user device 130. A network 140 may connect the data analysis system 110, the one or more data sources 120, and/or the end-user device 130.

The data analysis system 110 may be a computing device, such as a back-end server. The data analysis system 110 may include components that enable data analysis functions and practical applications thereof, such as alerting to inconsistent results of clinical trials through comparison to data stored in EMRs. The data analysis system 110 may use EMRs to conduct virtual clinical trials to study the effects of various ingredients and activities on patient health. The results can be used as source information for clinical trial planning and reporting, or may be used to assess the results of actual clinical trials.

The one or more data sources 120 may be computing devices and/or storage devices configured to supply data to the data analysis system 110. In one example, the one or more data sources 120 includes a medical records database 125 storing a plurality of EMRs. In at least some embodiments, the EMRs may provide the data analysis system 110 with information regarding patient medical histories, including symptoms and outcomes. The EMRs may be enhanced through techniques such as natural language processing and machine learning classifiers. For example, a system may perform natural language processing of clinical narrative notes to provide organized data to an EMR from an unstructured format. Moreover, a classifier developed through machine learning may analyze an EMR to add a medical status or condition to the patient medical history. For instance, a classifier for subjective issues such as pain or diseases that are under-documented may be developed and used to enhance the EMRs. The enhanced EMRs may be stored in the medical records database 125 and used in one or more disclosed methods to alert to results of a virtual clinical trial.

In some embodiments, the one or more data sources 120 may further include scientific literature documents, such as news sources, medical journals, legal texts, websites, books, etc. The scientific literature documents may include reports, studies, tests, trials, etc., that provide associations between patient information and medical outcomes, such as symptoms, family histories, skin characteristics, etc., and the development of a skin condition.

The end-user device 130 may be a computing device (e.g., a desktop or laptop computer, mobile device, etc.). The end-user device 130 may communicate with the data analysis system 110 to receive information and provide feedback related a skin condition assessment. In some embodiments, the end-user device 130 may include a user interface 135 enabling a user to view information such as the results of a skin condition assessment and patient or physician suggestions for future actions. In some embodiments, the user interface 135 may be associated with a medical decision support system (MDSS) that provides recommendations regarding treatment options to a clinical user.

The network 140 may be a local or global network and may include wired and/or wireless components and functionality which enable internal and/or external communication for components of the healthcare data environment 100. The network 140 may be embodied by the Internet, provided at least in part via cloud services, and/or may include one or more communication devices or systems which enable data transfer to and from the systems and components of the healthcare data environment 100.

In accordance with some exemplary embodiments, the data analysis system 110, data source(s) 120, end-user device 130, or the related components include logic implemented in specialized hardware, software executed on hardware, or any combination of specialized hardware and software executed on hardware, for implementing the healthcare data environment 100 or related components. In some exemplary embodiments, the data analysis system 110 or any of its components may be or include the IBM Watson system available from International Business Machines Corporation of Armonk, New York, which is augmented with the mechanisms of the illustrative embodiments described hereafter.

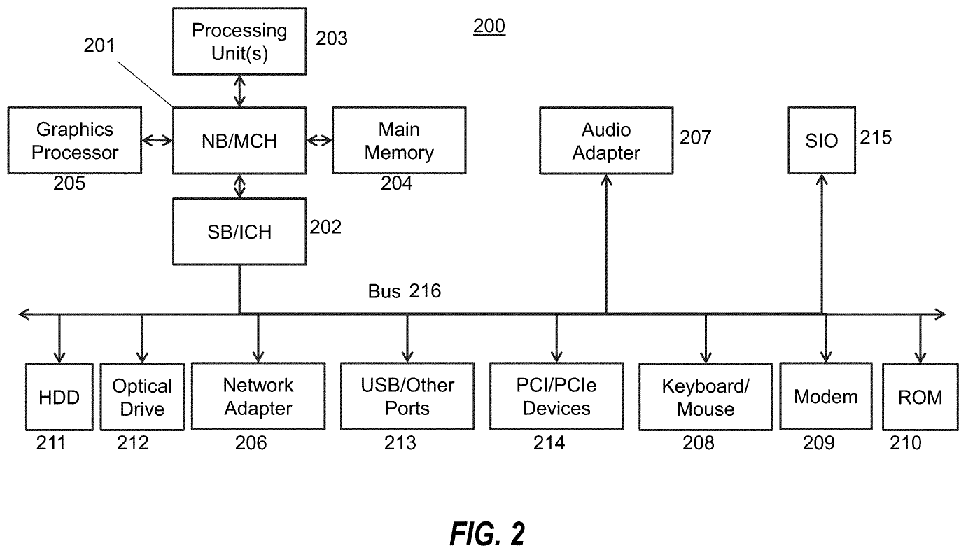

FIG. 2 is a block diagram of an example data processing system 200 in which aspects of the illustrative embodiments are implemented. Data processing system 200 is an example of a computer in which computer usable code or instructions implementing the process for illustrative embodiments of the present invention are located. In one embodiment, FIG. 2 represents the data analysis system 110, which implements at least some of the aspects of the healthcare data environment 100 described herein.

In the depicted example, data processing system 200 can employ a hub architecture including a north bridge and memory controller hub (NB/MCH) 201 and south bridge and input/output (I/O) controller hub (SB/ICH) 202. Processing unit 203, main memory 204, and graphics processor 205 can be connected to the NB/MCH 201. Graphics processor 205 can be connected to the NB/MCH 201 through an accelerated graphics port (AGP).

In the depicted example, the network adapter 206 connects to the SB/ICH 202. The audio adapter 207, keyboard and mouse adapter 208, modem 209, read only memory (ROM) 210, hard disk drive (HDD) 211, optical drive (CD or DVD) 212, universal serial bus (USB) ports and other communication ports 213, and the PCI/PCIe devices 214 can connect to the SB/ICH 202 through bus system 216. PCI/PCIe devices 214 may include Ethernet adapters, add-in cards, and PC cards for notebook computers. ROM 210 may be, for example, a flash basic input/output system (BIOS). The HDD 211 and optical drive 212 can use an integrated drive electronics (IDE) or serial advanced technology attachment (SATA) interface. The super I/O (SIO) device 215 can be connected to the SB/ICH 202.

An operating system can run on processing unit 203. The operating system can coordinate and provide control of various components within the data processing system 200. As a client, the operating system can be a commercially available operating system. An object-oriented programming system, such as the Java™ programming system, may run in conjunction with the operating system and provide calls to the operating system from the object-oriented programs or applications executing on the data processing system 200. As a server, the data processing system 200 can be an IBM® eServer™ System p® running the Advanced Interactive Executive operating system or the Linux operating system. The data processing system 200 can be a symmetric multiprocessor (SMP) system that can include a plurality of processors in the processing unit 203. Alternatively, a single processor system may be employed.

Instructions for the operating system, the object-oriented programming system, and applications or programs are located on storage devices, such as the HDD 211, and are loaded into the main memory 204 for execution by the processing unit 203. The processes for embodiments of the website navigation system can be performed by the processing unit 203 using computer usable program code, which can be located in a memory such as, for example, main memory 204, ROM 210, or in one or more peripheral devices.

A bus system 216 can be comprised of one or more busses. The bus system 216 can be implemented using any type of communication fabric or architecture that can provide for a transfer of data between different components or devices attached to the fabric or architecture. A communication unit such as the modem 209 or network adapter 206 can include one or more devices that can be used to transmit and receive data.

Those of ordinary skill in the art will appreciate that the hardware depicted in FIG. 2 may vary depending on the implementation. For example, the data processing system 200 includes several components which would not be directly included in some embodiments of the data analysis system 110. However, it should be understood that a data analysis system 110 may include one or more of the components and configurations of the data processing system 200 for performing processing methods and steps in accordance with the disclosed embodiments.

Moreover, other internal hardware or peripheral devices, such as flash memory, equivalent non-volatile memory, or optical disk drives may be used in addition to or in place of the hardware depicted. Moreover, the data processing system 200 can take the form of any of a number of different data processing systems, including but not limited to, client computing devices, server computing devices, tablet computers, laptop computers, telephone or other communication devices, personal digital assistants, and the like. Essentially, data processing system 200 can be any known or later developed data processing system without architectural limitation.

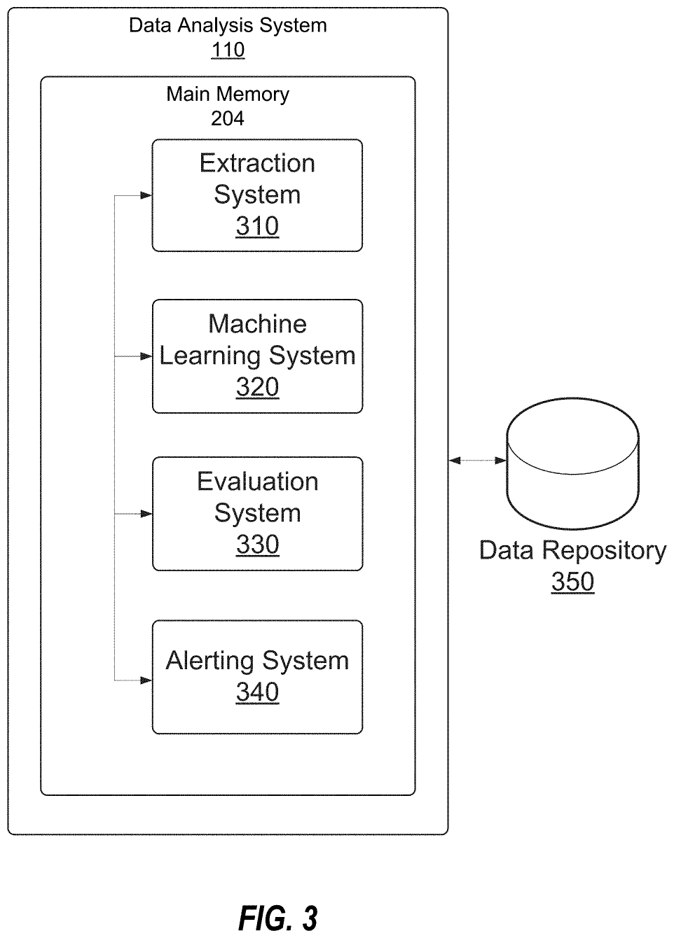

FIG. 3 illustrates an exemplary embodiment of the data analysis system 110. In an exemplary embodiment, the data analysis system 110 includes an extraction system 310, a machine learning system 320, an evaluation system 330, and an alerting system 340. These subsystems of the data analysis system 110 may be components of a single device, or may be separated devices connected to each other (e.g., via the network 140). In some embodiments, the data analysis system 110 may further include and/or be connected to one or more data repositories 350.

The extraction system 310 may be a computing device or component (e.g., software or hardware engine or module) configured to extract data from the one or more data sources 120. In one embodiment, the extraction system 310 may be configured to extract data from EMRs stored in the medical records database 125. The extraction system 310 may be configured to extract information from structured and unstructured data. For example, the extraction system 310 may process clinical narrative notes (an example of unstructured data) to extract indicators related to medical conditions, including but not limited to skin conditions. In another example, the extraction system 310 may process classification codes (an example of structured data) for determinations of patient outcomes. In some embodiments, the extraction system 310 may be configured to extract information from scientific literature documents, including news sources, medical journals, legal texts, and the like. For example, the extraction system 310 may be configured to identify relevant associations between symptoms and a skin condition based on a medical journal article on the skin condition.

The extraction system 310 may be further configured in some embodiments to extract information about an individual patient. For example, the extraction system 310 may be configured to extract relevant symptom and condition information from an individual patient's EMR (e.g., stored in the medical records database 125). The extraction system 310 may extract indicators from clinical narrative notes, or other unstructured or structured data in an EMR. In some embodiments, the extraction system 310 may receive a particular EMR associated with a patient for data extraction and further analysis by data analysis system 110.

The extraction system 310 may be configured to perform natural language processing on data elements within the one or more data sources 120. For example, the extraction system 310 may perform natural language processing of clinical narrative notes to extract medical information about a patient. In another example, the extraction system 310 is configured to perform natural language processing and extract medical information from scientific literature documents. The extracted medical information may include indicators for medical conditions, such as symptoms, test results, physician observations, medications, family histories, skin characteristics (e.g., freckles, moles, birthmarks, color, etc.).

The machine learning system 320 may be a computing device or component (e.g., software or hardware engine or module) configured to use extracted information to develop a predictive model for one or more skin conditions. The machine learning system 320 calculates a probability that a patient is vulnerable to a particular skin condition based on indicators extracted from the patient's EMR. The machine learning system 320 may generate a predictive model, for example, by extracting indicators and outcomes from a large collection of publicly-available narrative sources or other medical records data to evaluate the indicators of a patient to determine whether they match a profile of someone with a particular skin condition to a certain threshold level. As an example, in one embodiment the machine learning system 320 is trained with data representing individuals with Eosinophilic Fasciitis. The data elements include a variety of covariates associated with the Eosinophilic Fasciitis patients, including age, gender, laboratory values, comorbidities (either related to skin conditions or other diseases), medications, laboratory values, etc. In addition to values measured for each laboratory observation, the machine learning system 320 is also capable of calculating how long before the diagnosis of Eosinophilic Fasciitis each laboratory value was measured (e.g., number of days). The machine learning system 320 is also capable of identifying a trend of increase or a decrease considering a series of measurements captured before the first diagnosis of Eosinophilic Fasciitis. The machine learning system 320 is trained with the enhanced set of covariates, i.e., lab values, lab slopes, durations before first diagnosis of Eosinophilic Fasciitis, etc. When a new patient (e.g., a patient that does not have Eosinophilic Fasciitis) receives care, the machine learning system 320 can determine whether the patient has an EMR profile similar to patients who develop Eosinophilic Fasciitis at a later stage. A high level of similarity will result an alert notifying that the patient is at a high risk to develop Eosinophilic Fasciitis.

The machine learning system 320 may be configured to develop a predictive model that produces a probability that a given patient will develop a particular skin condition. For example, the machine learning system 320 may calculate a numerical probability based on similarity matching to extracted data from EMRs of patients that did and did not develop that skin condition. In some embodiments, the machine learning system 320 may be configured to calculate a probability profile that includes a probability of developing a skin condition over time (e.g., Current Age: 10%, Age 35: 15%, Age 45: 20%, etc.). The machine learning system 320 may be further configured to receive or generate a threshold for deciding how to classify a patient (e.g., as either having or not having a particular skin condition or class of skin conditions).

In some embodiments, the machine learning system 320 may also be configured to produce a successful treatment suggestion model based on extracted EMR data. For example, for a given skin condition, the machine learning system 320 may review EMR data of patients that recover or experience the least severity of symptoms and develop a suggestion model for providing patient actions that may be taken to lessen the likelihood of developing a skin condition.

The evaluation system 330 may be a computing device (e.g., software or hardware engine or module) configured to use a predictive model developed by the machine learning system 320 to assess the condition of a patient and determine one or more predictive results. For example, the evaluation system 330 may receive extracted information from a patient's EMR and evaluate apply a machine learning model for a plurality of skin conditions to obtain a result, such as a probability or assessment of the patient's likelihood of having or developing the skin condition. In some embodiments, the evaluation system 330 is configured to receive extracted EMR data for a patient (or class of patients), enter the EMR data into a predictive model, receive output, and evaluate the output for contextual meaning. For example, the evaluation system 330 may compare a probability from a predictive model to a stored threshold to assess whether the patient is classified as having the skin condition. In some embodiments, the evaluation system 330 may be configured to make judgements based on degrees of probability and/or the severity of the skin condition and categorize each potential skin condition into classifications such as “high risk,” “medium risk,” “low risk,” “no risk,” “has condition,” etc.

The alerting system 340 may be a computing device (e.g., software or hardware engine or module) configured to provide information to end-user device 130 based on results determined by the evaluation system 330. For instance, the alerting system 340 may provide an alert to the end-user device 130 identifying the probability that a particular patient will develop a particular skin condition based on results from an associated predictive model developed by the machine learning system 320. In some embodiments, the alerting system 340 may provide information to end-user device 130 that the end-user device 130 displays through user interface 135. The information may include probabilities related to skin conditions, suggested actions, process information (e.g., relevant patient indicators), etc.

The data repository 350 may be a database configured to store data. The data repository 350 may be configured to receive data from the extraction system 310 and/or from one or more data sources 120 and store the data according to appropriate storage protocols. In some embodiments, the data repository 350 receives data from the data analysis system 110, such as from the extraction system 310. In other embodiments, the data repository 350 receives data from the one or more data sources 120 and is a data supply for the data analysis system 110.

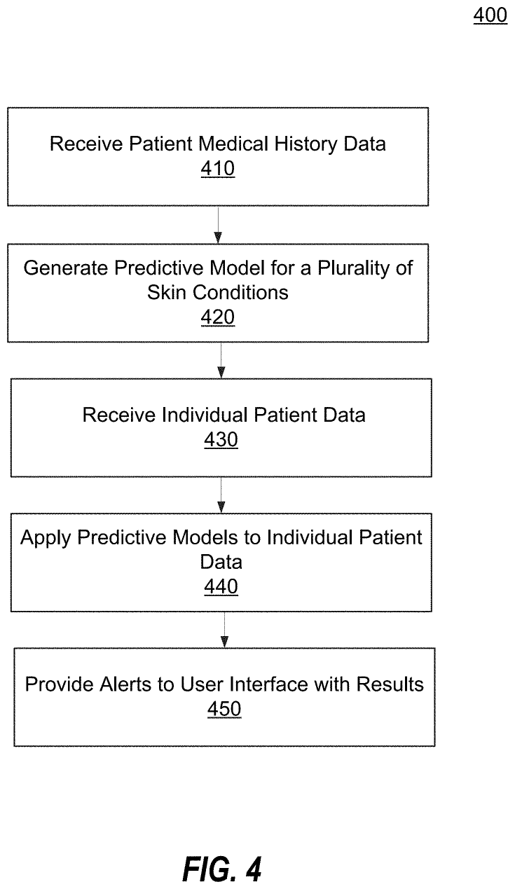

FIG. 4 is a flowchart of an exemplary process for alerting to an assessment of skin condition and vulnerabilities. The data analysis system 110 may perform one or more steps of the process 400 in order to use information from data source(s) 120 (e.g., EMR data) to develop and use predictive models to provide an assessment of a selected patient or class of patients.

In step 410, the data analysis system 110 receives patient medical history data. For example, the extraction system 310 may receive extract information from the one or more data sources 120, such as medical records database 125. The extraction system 310 may process structured and unstructured data from EMRs to identify indicators of skin and other medical conditions. In one example, the extraction system 310 may process clinical narrative notes using natural language processing. In another example, the extraction system 310 may identify skin conditions from EMRs (e.g., stored in the form of one or more medical classification codes (e.g., ICD codes).

In step 420, the data analysis system 110 generates predictive models for a plurality of skin conditions. For example, the machine learning system 320 may use the patient medical history data to develop machine learning algorithms that predict the likelihood that a patient has a skin condition given a medical history profile. In some embodiments, the predictive model may be used to produce a probability that a patient will develop one or more skin conditions. In some embodiments, the machine learning system 320 may generate a predictive model for each of a plurality of skin diseases, including, for example, Darier's Disease, Lichen Planus Actinicus, Polymorphic Light Eruption, melanoma, etc.

Consistent with disclosed embodiments, the data analysis system 110 may produce a predictive model by reviewing indicators (e.g., symptoms, test results, physician observations, medications, family histories, skin characteristics) in relation to one or more outcomes, such as data indicating that the patient developed or did not develop the skin condition. The machine learning system 320 may utilize supervised or unsupervised learning to develop one or more algorithms that produce assessment results, such as probabilities.

In some embodiments, the machine learning system 320 may develop one or more suggestion models for providing effective suggestions for a patient that is vulnerable to a particular skin condition. The suggestion model may include one or more algorithms that match predicted skin conditions with one or more actions for effectively addressing the condition based on successful past actions of other patients found in the one or more data sources 120.

In step 430, the data analysis system 110 receives individual patient data. For example, the evaluation system 330 may receive a request to evaluate a patient based on an

EMR associated with the patient. The data analysis system 110 may receive the EMR and/or the extraction system 310 may extract medical history information from the EMR. For example, the extraction system 310 may extract indicators (e.g., symptoms, test results, physician observations, medications, family histories, skin characteristics, etc.) from the EMR. In some embodiments, the extraction system 310 may perform natural language processing of clinical narrative notes to extract the indicators associated with a patient. For example, the extraction system 310 may identify that a physician's narrative note from a patient physical states that the patient “reports itchy, dry skin” or that the physician “observed moles and freckles.” The extraction system 310 may combine information taken from unstructured data (e.g., the clinical narrative notes) with structured data, such as test results, billing codes, symptom charts, medical history charts, etc.

In other embodiments, the evaluation system 330 may receive patient data from the end-user device 130. For example, the end-user device 130 may produce a user interface 135 that includes fields for a user to input relevant information, such as by answering questions, providing documents, etc. The end-user device 130 may provide the patient data to the data analysis system 110. In this way, the data analysis system 110 may provide a skin condition assessment tool for a use on an end-user device (e.g., through a mobile application, website, software program, MDSS, etc.).

In step 440, the data analysis system 110 applies one or more predictive models to the individual patient data. For example, the evaluation system 330 may input the patient data into a predictive model and obtain assessment results. The results may include an assessment by the data analysis system 110 of the likelihood that the person associated with the received patient data will develop a particular skin condition (e.g., a skin condition associated with the predictive model).

In some embodiments, the evaluation system 330 may apply the patient data and obtain a probability that the patient will develop the skin condition. In some aspects, the probability may be in the form of a classification (e.g., has condition, does not have condition, vulnerable, expected to develop condition, etc.). The evaluation system 330 may determine the classification by comparing a numerical probability to a threshold. In another embodiment, the evaluation system 330 may use the predictive models to produce a vulnerability score. The vulnerability score may be a representation of the patient's likelihood of developing the skin condition in their lifetime. In still other embodiments, the evaluation system 330 may develop a probability profile that includes a probability of the patient developing the skin condition at different points in their life (e.g., different ages).

In some embodiments, the evaluation system 330 may also determine one or more proposed actions for the patient and/or physician based on the results of the predictive model. Potential actions could be simplistic, such as “Recommended action: increase physical activity,” “Recommended action: decrease exposure to sun,” etc. More advanced actions could be educating the patient on potential side effects of medications he or she may be asked to take if the patient actually develops the skin disease. Such recommendations may be tailored to the individual patient and include factors extracted from the EMR record, such as allergies.

In step 450, the data analysis system 110 may provide an alert based on the result of the evaluation system 330. For example, the alerting system 340 may provide an alert to the end-user device 130 indicating the results of the predictive modeling for various skin conditions. For example, the alerting system 340 may provide a list of skin conditions that the patient has and/or does not have. In another example, the alerting system 340 may provide a probability or vulnerability score for a plurality of skin conditions. The alerting system 340 provides the results to the end-user device 130 to be presented to a user (e.g., through user interface 135).

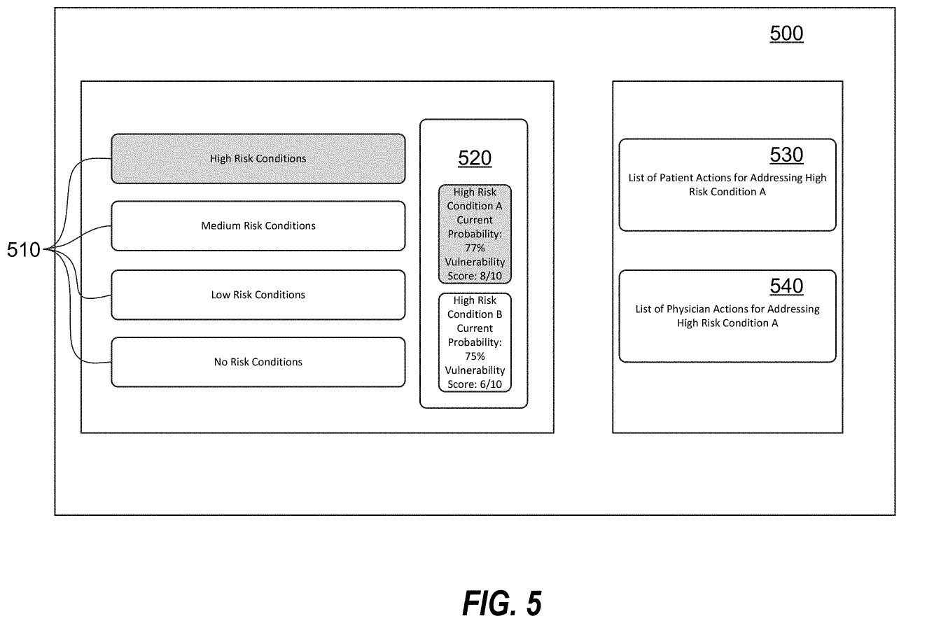

FIG. 5 is an exemplary depiction of a user interface 500 for displaying the results of a skin condition assessment performed by the data analysis system 110. The user interface 500 may correspond to the user interface 135 of the end-user device 130. For example, the user interface 500 may be displayed via a screen associated with a mobile device, laptop, desktop, etc. The user interface 500 comprises various display elements that provide feedback to a user regarding the results of the assessment.

In one example, the user interface 500 includes classifications 510 of skin conditions based on results of associated predictive models (e.g., a predictive model for each skin condition). For instance, the classifications may include high risk conditions, medium risk conditions, low risk conditions, and no risk conditions. The user interface 500 may further include a listing 520 of the conditions in each classification, depending on a selected classification (e.g., high risk conditions). In some embodiments, the listing 520 may include a current probability indicating the likelihood that the patient has the skin condition. In some embodiments, the listing 520 may also include a vulnerability score indicating the likelihood that the patient will develop the skin condition in the future.

The user interface 500 may also include listings 530 and 540 for suggested actions that may be taken by the patient and/or the physician, respectively, depending on a selected condition (e.g., high risk condition A). For example, the listing 530 may include suggestions such as avoiding sunlight during certain times of day, wearing sunblock, over-the-counter products, dietary suggestions, etc. The listing 540 may include, for example, suggested diagnosis to consider, prescription medication, or other medical advice to provide to the patient. The information in the listings 530, 540 may be received based on stored suggestions for a particular condition, or may be specifically tailored based on the patient's medical history.

The disclosed embodiments provide a system and associated methods for leveraging a large dataset having medical history information to develop predictive models that provide an early alert to skin conditions. Skin conditions are often treatable but may be easily aggravated when not properly addressed. For instance, many skin conditions are exacerbated by sun exposure, but the individual is unaware that they even have the condition. Therefore, a system that uses known indicators as input into a predictive model trained on known outcomes would help patients detect or determine that they have a condition early enough to address it through remedial actions, such as by reducing sun exposure.

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

The system and processes of the Figures are not exclusive. Other systems, processes and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112, sixth paragraph, unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

What is claimed is:

1. A computer-implemented method for alerting to the results of a skin condition assessment in a data processing system comprising a processing device and a memory comprising instructions which are executed by the processor, the method comprising:
receiving medical history information for a plurality of patients;
generating a predictive model for a skin condition based on the medical history information for a plurality of patients;
receiving medical data for an individual patient;
applying the predictive model for the skin condition to the medical data for the individual patient; and
alerting to results of the predictive model by providing the results to a user interface of an end-user device, wherein the results include an assessment of the likelihood that the individual patient will develop the skin condition.

2. The method of claim 1, further comprising extracting one or more indicators from the medical history information for the plurality of patients.

3. The method of claim 2, wherein the one or more indicators comprise one or more of symptoms, test results, physician observations, medications, family histories, skin characteristics.

4. The method of claim 2, wherein extracting the one or more indicators comprises performing natural language processing of clinical narrative notes.

5. The method of claim 1, further comprising extracting one or more outcomes from the medical history information for the plurality of patients.

6. The method of claim 5, wherein extracting the one or more outcomes comprises identifying the presence of absence of a classification code associated with the skin condition.

7. The method of claim 1, further comprising extracting one or more indicators and one or more outcomes from the medical history information for the plurality of patients, and wherein generating a predictive model for a skin condition comprises applying a machine learning algorithm on the one or more indicators and one or more outcomes.

8. The method of claim 1, wherein generating a predictive model for a skin condition comprises generating a plurality of predictive models, each associated with a different skin condition.

9. The method of claim 1, wherein receiving medical data for the patient comprises extracting information from an electronic medical record associated with the patient.

10. The method of claim 9, wherein extracting information comprises performing natural language processing of clinical narrative notes.

11. The method of claim 1, wherein the results comprise a probability that the patient will develop the skin condition.

12. The method of claim 11, wherein the results further comprise a probability profile that includes a probability of the patient developing the skin condition at different points in time.

13. The method of claim 1, wherein the results comprise a classification of the assessment.

14. The method of claim 13, wherein the classification is selected from labels including has condition, does not have condition, vulnerable to condition, or expected to develop condition.

15. The method of claim 13, wherein the user interface comprises a listing of conditions associated with the classification.

16. The method of claim 1, further comprising generating a suggestion model based on the medical history information, the suggestion model configured to provide one or more suggested actions for addressing a skin condition.

17. The method of claim 16, wherein the results include one or more suggested actions for the patient or a physician based on the assessment.

18. The method of claim 17, wherein the one or more suggested actions comprises guidelines for exposure to sunlight.

19. A data analysis system for alerting to an assessment of one or more skin conditions, the data analysis system comprising:
an extraction system for extracting one or more indicators and one or more outcomes related to a plurality of skin conditions from electronic medical records containing medical history information for a plurality of patients;
a machine learning system for generating a predictive model for each of the plurality of skin conditions based on the extracted one or more indicators and one or more outcomes;
an evaluation system for receiving medical data for an individual patient and applying each predictive model to the medical data for the individual patient; and
an alerting system for providing results of each predictive model to a user interface of an end-user device, wherein the results include an assessment of the likelihood that the individual patient will develop each skin condition.

20. A computer program product for alerting to the results of an assessment of a skin condition, the computer program product comprising a computer readable storage medium having program instructions embodied therewith, the program instructions executable by a processor to cause the processor to:
receive medical history information for a plurality of patients;
generate a predictive model for a skin condition based on the medical history information for a plurality of patients;
receive medical data for an individual patient;
apply the predictive model for the skin condition to the medical data for the individual patient; and
alert to results of the predictive model by providing the results to a user interface of an end-user device, wherein the results include an assessment of the likelihood that the individual patient will develop the skin condition.
